(19) United States
(12) Patent Application Publication
(10) Pub. No.: US 2021/0123052 A1
(43) Pub. Date: Apr. 29, 2021

(54) METHODS FOR THE IN VIVO PRODUCTION OF SINGLE STRANDED DNA AND USES THEREOF

(71) Applicant: The Trustees of Columbia University in the City of New York, New York, NY (US)

(72) Inventors: Alejandro Chavez, New York, NY (US); Schuyler Melore, New York, NY (US)

(73) Assignee: The Trustees of Columbia University in the City of New York, New York, NY (US)

(21) Appl. No.: 17/080,240

(22) Filed: Oct. 26, 2020

Related U.S. Application Data
(60) Provisional application No. 62/926,110, filed on Oct. 25, 2019.

(51) Int. Cl. C12N 15/113; C12N 9/22

(52) U.S. Cl. CPC C12N 15/113; C12N 2310/20; C12N 2800/80; C12N 9/22

(57) ABSTRACT

The present disclosure relates generally to oligonucleotide production. In particular it relates to in vivo single stranded DNA (ssDNA) production.

### RELATED APPLICATION DATA

This application claims priority to U.S. Provisional Application No. 62/926,110 filed on Oct. 25, 2019, which is hereby incorporated herein by reference in it its entirety for all purposes.

### FIELD OF THE INVENTION

The present disclosure relates generally to oligonucleotide production. In particular it relates to in vivo single stranded DNA (ssDNA) production.

### BACKGROUND

ssDNA oligonucleotides (oligos) are multi-faceted molecules that can be applied to a variety of applications including genome editing, CRISPR-cas9, aptamer synthesis, tape recorder methods, antisense oligo production, gene target delivery, and therapeutic treatments. ssDNA is preferred due to its low toxicity profiles, increased stability, and easy modification. Traditionally, chemical and enzymatic methods such as systematic evolution of ligands by exponential enrichment (SELEX), are utilized for synthesizing ssDNA in vitro. However, conventional methods are limited by low affinity, low efficiency, low yield, maximum oligo size, and high costs.

There exists a need to eliminate the above identified short-comings.

### SUMMARY OF THE DISCLOSURE

The present invention discloses methods of producing ssDNA oligos in vivo using HIV reverse transcriptase (RT) and an RNA target. In one embodiment, this method introduces a DNA construct into cells that is incorporated into the endogenous RNA polymerase machinery, and then converted back into ssDNA using transfected HIV RT. This approach efficiently enables production of large quantities of ssDNA in mammalian cells, allowing for applications in disease gene correction, genome editing, CRISPR-cas9, aptamer design, antisense oligo production, targeted delivery, and other potential therapeutic interventions.

Accordingly, in a first aspect, the present invention provides a method of producing a single stranded DNA oligonucleotide in vivo, comprising: introducing a DNA construct encoding a sequence of interest into a cell, wherein the sequence of interest is operably linked to a promoter sequence recognizable by the cell's endogenous RNA polymerase machinery; introducing a reverse transcriptase fused to a nuclear localization signal and a RNA binding protein into the cell; wherein the sequence of interest is fused to a motif that is recognized by the RNA binding protein after being transcribed; thereby producing the single stranded DNA oligonucleotide.

In various embodiments of the first aspect of the invention delineated herein, the method further comprises introducing a DNA cleaving enzyme into the cell to cleave a target locus, wherein the single stranded DNA oligonucleotide repairs the DNA by homology-directed repair (HDR).

In various embodiments of the first aspect of the invention delineated herein, the DNA cleaving enzyme is Cas9, a Zinc Finger enzyme, an Activator-Like Effector Nuclease (TALEN) enzyme, or any one of the CRISPR-associated DNA cleaving enzymes.

In various embodiments of the first aspect of the invention delineated herein, the reverse transcriptase is a human immunodeficiency virus reverse transcriptase (HIV RT).

In various embodiments of the first aspect of the invention delineated herein, the RNA binding protein is a MS2 coat protein (MCP).

In various embodiments of the first aspect of the invention delineated herein, the RNA binding protein is a PP7 coat protein (PCP).

In various embodiments of the first aspect of the invention delineated herein, the RNA binding protein is fused to the N-terminus, the C-terminus, or both termini of the reverse transcriptase.

In various embodiments of the first aspect of the invention delineated herein, the DNA construct further comprises an HIV terminator-binding site (HTBS).

In various embodiments of the first aspect of the invention delineated herein, the motif is a MS2 RNA motif.

In various embodiments of the first aspect of the invention delineated herein, the motif is a PP7 RNA motif.

In various embodiments of the first aspect of the invention delineated herein, the cell is a mammalian cell.

In various embodiments of the first aspect of the invention delineated herein, the method further comprises DNA barcoding biomolecules within cells using the single stranded DNA oligonucleotide.

In various embodiments of the first aspect of the invention delineated herein, the biomolecule is a protein.

In various embodiments of the first aspect of the invention delineated herein, a plurality of different DNA constructs are introduced into a population of cells, such that each cell receives a single DNA construct, to thereby produce a plurality of ssDNA oligonucleotides.

In various embodiments of the first aspect of the invention delineated herein, the method further comprises screening the plurality of ssDNA oligonucleotides to identify ssDNA oligonucleotides having one or more desired property.

In various embodiments of the first aspect of the invention delineated herein, the method further comprises screening the plurality of ssDNA oligonucleotides to identify one or more ssDNA oligonucleotide that functions as an antisense oligonucleotide to knockdown a target gene of interest.

In various embodiments of the first aspect of the invention delineated herein, the method further comprises screening the plurality of ssNDA oligonucleotides to identify one or more ssDNA oligonucleotide that functions as an aptamer to bind to a target protein of interest.

### BRIEF DESCRIPTION OF THE FIGURES

FIG. 1. Overview of the present disclosure and some of its applications. HIV RT is expressed within mammalian cells and reverse transcribes a programmable RNA target to produce ssDNA oligonucleotides. These oligos can then be used in concert with Cas9 for precision genome editing, as an antisense technology that stimulates RNAseH-mediated degradation of target mRNAs, as DNA aptamers that modulate the activity of a protein of interest, or in other applications of interest.

FIG. 2A, FIG. 2B. Components of the HIV RT-based system. FIG. 2A) Plasmid encoding the RT expresses the two subunits, p66 and p51, of the HIV RT, both of which contain a nuclear localization signal (NLS). The two subunits heterodimerize to form functional HIV RT. FIG. 2B) Plasmid encoding the RNA target expresses the RNA target under the control of an RNA Polymerase III promoter, U6. The target is composed of a fusion of the tRNA^{Lys3}, which acts as a primer for reverse transcription in the native HIV life cycle, to a programmable sequence of interest. This RNA construct is deemed the HIV Terminator-Binding Site (HTBS) since it contains the native HIV primer binding site, a transcriptional terminator for the U6 promoter, and the programmable sequence of interest.

FIGS. 3A, 3B, 3C. Process by which HIV RT reverse transcribes its target RNA. FIG. 3A) The heterodimeric RT binds to the HTBS. FIG. 3B) The RT extends from the primer binding site within the HTBS and converts the programmable target RNA to ssDNA, destroying the RNA template with its RNAseH domain as it progresses. FIG. 3C) the target RNA has now been fully reverse transcribed into ssDNA that can be used for downstream applications.

FIG. 4. Production of ssDNA oligonucleotides by wild type and MS2 or PP7-modified HIV RT constructs. Cells were transfected with HTBS constructs and RT. Negative controls were transfected with iRFP instead of RT. HIV RT modified to use the PP7 or MS2 systems show an approximately 2 or 3 fold increase, respectively, in relative ssDNA oligonucleotide production over the unmodified system. Data are shown as the mean±s.e.m (n=2 independent transfections).

FIG. 5. Production of ssDNA oligonucleotide by wild type and MS2 or PP7-modified HIV RT constructs. Cells were transfected with HTBS constructs and RT. Negative controls were transfected with iRFP instead of RT. HIV RT with MCP fused to both subunits (p66 and p51) shows an approximately 2 fold increase in relative ssDNA oligonucleotide production over HIV RT with MCP fused to just a single subunit. Data are shown as the mean±s.e.m (n=2 independent transfections).

FIGS. 6A, 6B. An application of oligo production for precision genome editing. FIG. 6A) HIV RT produces many short ssDNA donor oligonucleotides homologous to the sequence of BFP, but containing the H66Y amino acid substitution, while Cas9 cuts the BFP locus. FIG. 6B) Once a double-stranded break is induced, proximal donor oligos can be used as a repair template for HDR. Cells that use the donor oligos for repair will now express GFP instead of BFP.

FIG. 7. Oligo production by HIV RT increases HDR rates in 293T cells. Cells were transfected with BFP, Cas9, a gRNA against BFP, HTBS, and HIV RT. Negative controls were transfected with iRFP instead of HIV RT. The BFP to GFP conversion experiment was performed as described above and quantified the result by flow cytometry. It was found that our MCP-MS2 system led to a 2 fold increase in HDR rates over the negative control. Data are shown as the mean s.e.m (n=3 independent transfections).

### DETAILED DESCRIPTION

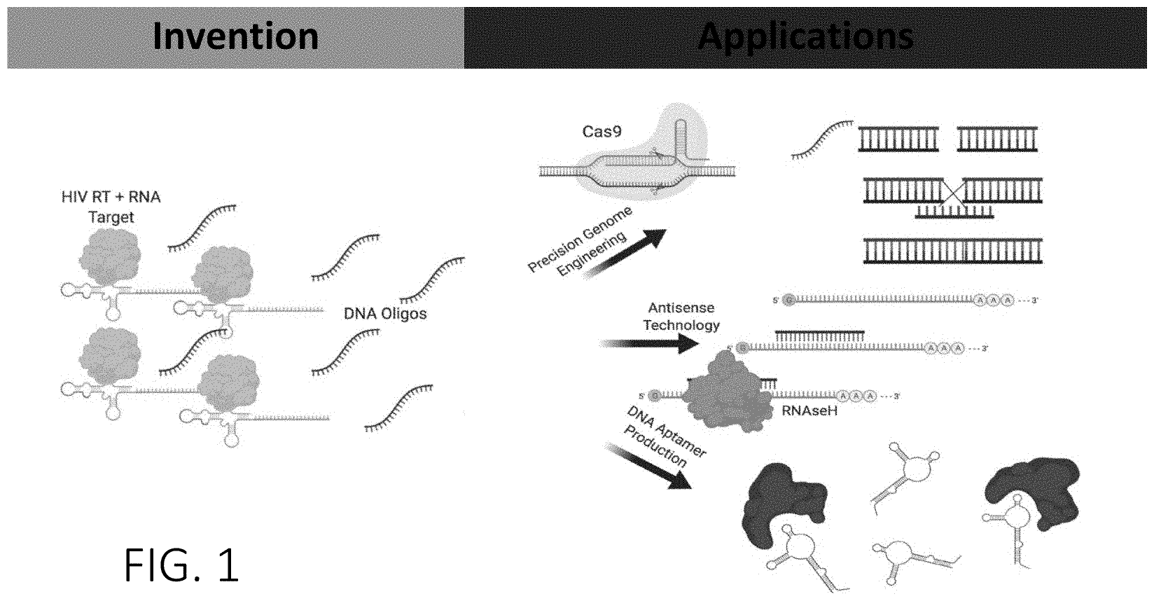

The present disclosure is directed, at least in part, to methods and systems for the production of single stranded DNA (ssDNA) of programmable sequence in vivo and uses thereof (see FIG. 1). In some embodiments, the disclosure provides methods for high throughput modeling of many different genetic variations within a pooled population of cells. The methods and systems of the disclosure greatly decrease the time, cost, and effort required to model and elucidate the role of genetic variants suspected to play a role in a particular disease. The methods and systems of the disclosure can also be used for several additional applications, including, but not limited to, genome engineering, aptamer production and screening, in vivo tape recorder methods, antisense oligo production and screening, and DNA barcoding biomolecules, e.g., proteins, within cells.

Accordingly, in some aspects, the present disclosure is directed to methods for producing ssDNA oligonucleotides in vivo. In some embodiments, in order to generate large amounts of ssDNA oligonucleotides in vivo, a DNA construct encoding a sequence of interest is introduced into cells of interest and is transcribed into RNA using the cell's endogenous RNA polymerase machinery. These RNA molecules are then converted back into ssDNA via the expression of a reverse transcriptase within the cells of interest. In some embodiments, the sequence of interest is fused to a motif, which after being transcribed into RNA using the cell's endogenous RNA polymerase machinery, is then recognized by a RNA binding protein. In some embodiments, the RNA binding protein is fused to a reverse transcriptase. In some embodiments, the cells of interest are mammalian cells. As a single DNA construct can generate thousands of RNAs, each of which can be converted back into DNA, the methods and systems described herein enable a large amount of ssDNA oligonucleotides to be produced from a single construct in vivo.

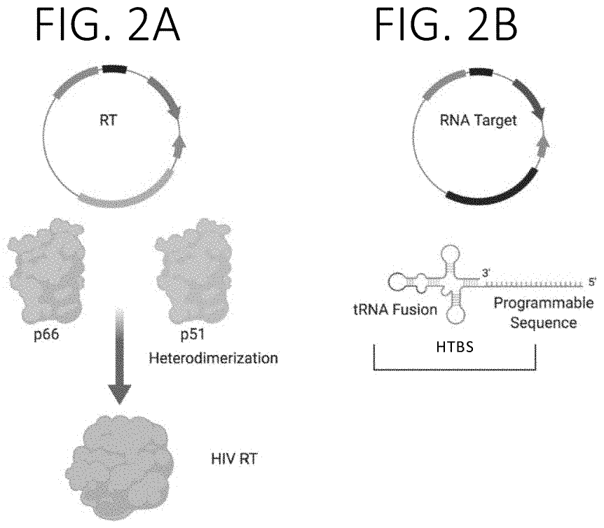

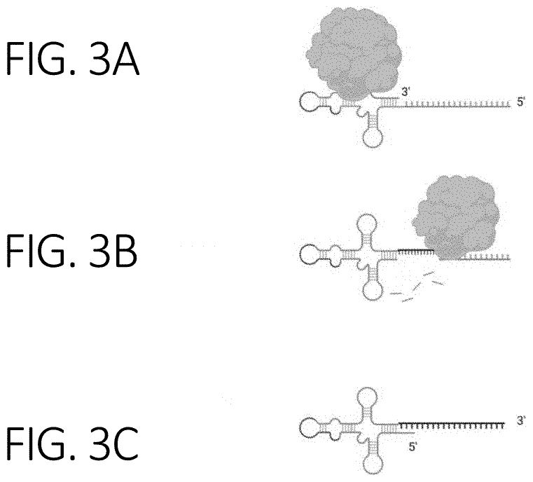

In some embodiments, the reverse transcriptase is a human immunodeficiency virus reverse transcriptase (HIV RT). In some embodiments, the HIV RT is modified to produce an HIV RT-based system that comprises a heterodimeric RT and an RNA target, which is referred to as an HIV terminator-binding site (HTBS) (see FIGS. 2A-2B and FIGS. 3A-3C). In some embodiments, the HIV RT-based system is modified to work in mammalian cells by, for example, adding nuclear localization signals (NLSes) to each of the subunits of HIV RT. In addition, in some embodiments, the HIV RT-based system is also modified by using one or more RNA binding protein, e.g., the MS2 and PP7 systems, to enhance recruitment of HIV RT to its target. These systems work to bring together a protein of interest with a sequence of interest by fusing one or more RNA binding protein, e.g., an MS2 coat protein (MCP) or PP7 coat protein (PCP), to the protein of interest, e.g., HIV RT, and including one or more RNA motif, e.g., the short MS2 or PP7 RNA motif, within the sequence of interest. The RNA binding protein, e.g., MCP or PCP, is then recruited to and tightly binds the sequence of interest containing the RNA motif, e.g., MS2 or PP7, respectively, bringing the protein of interest into close proximity with the sequence of interest.

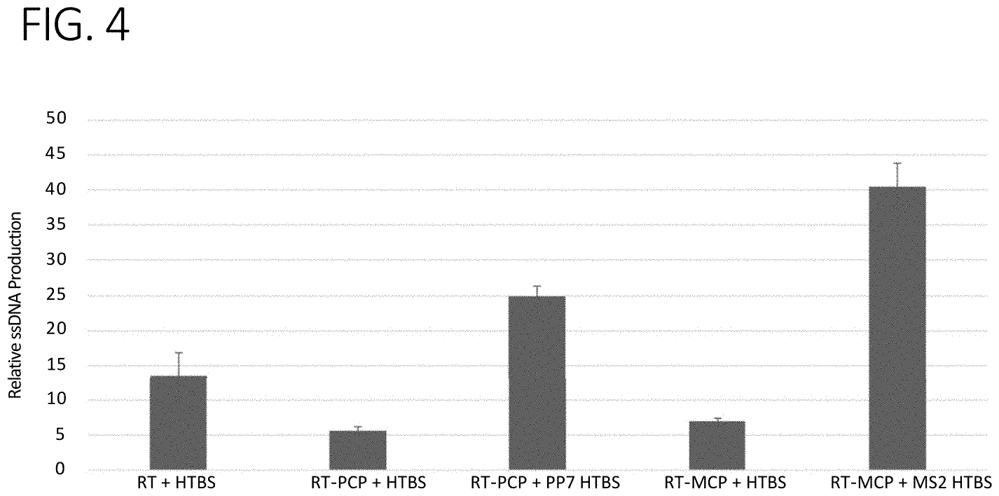

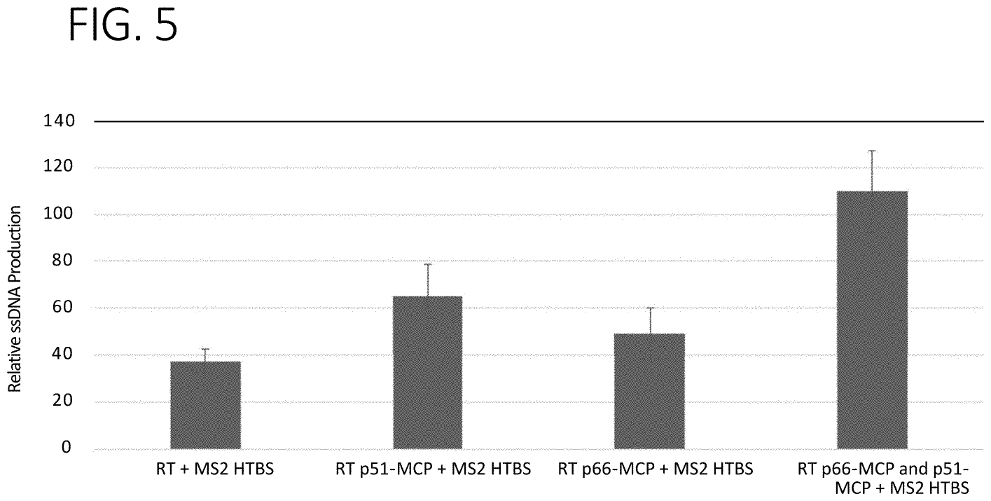

The inventors have shown that when MCP or PCP was fused to either the p66 or p51 subunit of HIV RT and MS2 or PP7 were included within the HTBS, about 3 fold or 2 fold increases in ssDNA production were observed, respectively (see FIG. 4). When MCP was fused to both subunits of the RT, an increase in ssDNA production of about 2 fold over cells that had MCP fused to just one of the subunits was observed (see FIG. 5).

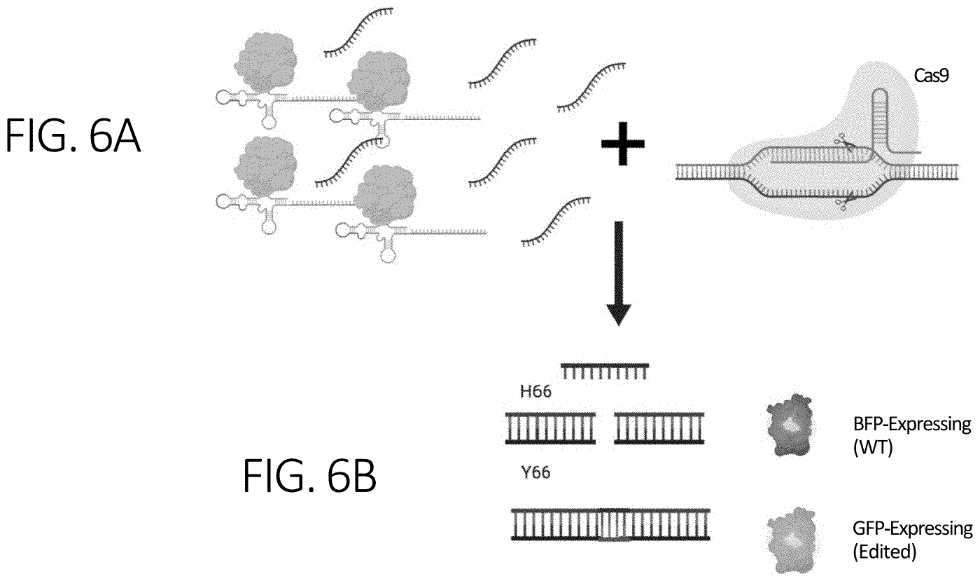

The present disclosure is also directed, at least in part, to methods of genome editing. In some embodiments, production of ssDNA oligonucleotides in the nucleus, based on the methods described in the present disclosure, can be used to create precision genome edits. For example, in one embodiment, a DNA cleaving enzyme cleaves a locus, and the ssDNA oligonucleotides produced by the methods disclosed herein are used for homology-directed repair (HDR) of the DNA (see FIGS. 6A-6B). In some embodiments, the DNA cleaving enzyme is, for example, Cas9, a Zinc Finger enzyme, an Activator-Like Effector Nuclease (TALEN) enzyme, or any one of the CRISPR-associated DNA cleaving enzymes.

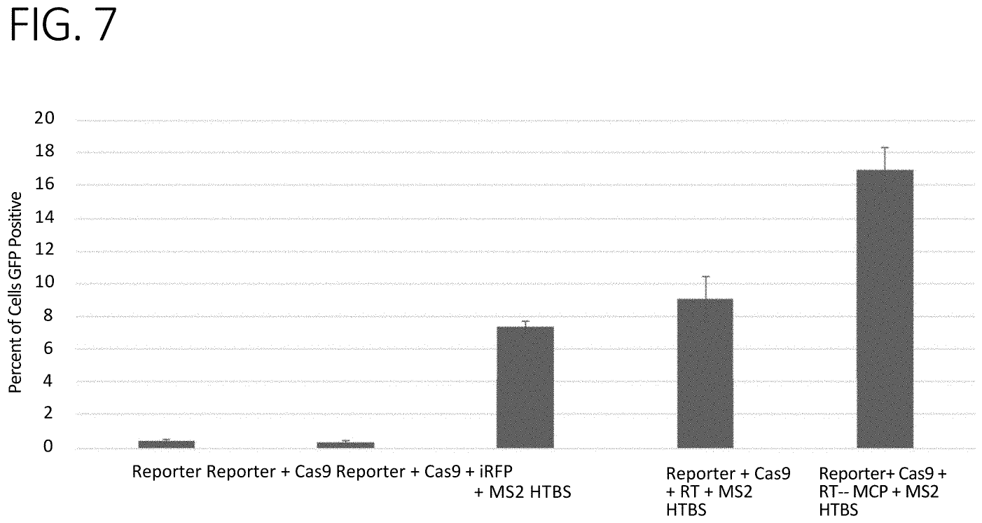

To test this hypothesis, Cas9 was used to cleave within BFP, a fluorescent protein, and HIV RT was used to create a 144 bp ssDNA donor that, if used for repair, would convert BFP to GFP (see FIGS. 6A-B6). It was found that using the methods disclosed herein for producing ssDNA in vivo, the rate of HDR was doubled as compared to cells that were not able to produce ssDNA (see FIG. 7), illustrating that the methods and systems disclosed herein are functional and useful in practical applications.

In some aspects, the methods and systems of the disclosure are used for applications, including, but not limited to, genome engineering, aptamer production, in vivo tape recorder methods, antisense oligo production, and DNA barcoding biomolecules, e.g., proteins, within cells.

In some embodiments, the methods and systems of the present disclosure are used as a screening tool to identify DNA oligonucleotides having desired properties. In some embodiments, the methods and systems of the disclosure are used to screen libraries, e.g., libraries of antisense oligonucleotides or aptamers, to identify those having desired properties.

The methods of the present disclosure enable expression of a single stranded DNA oligonucleotide of interest at high levels within cells. Therefore, the disclosure provides screening methods in which a library comprising a plurality, e.g., thousands, of different ssDNA-producing constructs can be generated and tested in parallel in order to screen for DNA oligonucleotides having one or more desired properties. In some embodiments, the library of constructs are delivered to a population of cells in such a way that each cell receives a single DNA construct (e.g., via low titer lentiviral transduction). Once introduced into the cell, the DNA constructs can express their encoded ssDNA via the methods disclosed herein.

In some embodiments, the methods of the invention can be used to screen for antisense oligonucleotides of interest. In order to screen for the ability of a given ssDNA oligonucleotide to function as an antisense oligonucleotide causing the targeted knockdown of a gene of interest, the population of cells can be stained with an antibody against the protein product of the targeted gene. Cells containing ssDNA oligonucleotides that are efficient antisense oligonucleotides will show reduced staining as compared to cells which contain ssDNA oligonucleotides that are poor antisense oligonucleotides. By using fluorescence-activated cell sorting (FACS), cells with reduced antibody staining can be sorted, and the prevalence of the various ssDNAs within this sorted population as compared to the initial unsorted pool can be determined. In doing so, ssDNA oligonucleotide which are effective antisense oligonucleotides against a given target gene can be identified, e.g., via sequencing. Alternatively, in some embodiments, if the targeted gene causes a growth phenotype when knocked down, the library of cells, each of which expresses a unique ssDNA, can be passaged. Over time, cells with the most potent antisense oligonucleotides will exhibit a growth defect and be depleted from the pool as the essential gene the antisense oligo targets will be lacking from those cells. The frequency of the various ssDNA-producing constructs at the start and end of the experiment can be sequenced to determine which ssDNA oligonucleotides were the most effective antisense oligonucleotides.

In other embodiments, similar to the approach for identifying antisense oligonucleotides against essential genes, as described herein, a screening strategy can also be used to identify DNA aptamers that inhibit proteins critical for cell growth. In some embodiments, in order to identify aptamers that bind to a particular protein in vivo, a yeast-3-hybrid-like approach can be used, in which a target protein is fused to a DNA binding domain. Cells expressing the DNA binding domain fused to the target protein are then infected with a library of DNA constructs, each of which expresses a unique DNA aptamer fused to an additional DNA sequence, which is able to recruit a transcriptional activation domain. If the aptamer interacts with the target protein, a synthetic transcription factor will be formed comprising a DNA binding domain derived from the target protein of interest and an activation domain provided by the aptamer. This synthetic transcription factor is then capable of activating the expression of a reporter gene (e.g., a yellow fluorescent protein). By using this reporter gene, aptamers of interest can be identified by isolating cells via FACS, followed by sequencing the identity of the ssDNA oligonucleotides within the cells.

In some embodiments, the approach can be used to create DNA-barcoded proteins. To do this cells are made to express a single stranded DNA molecule which then associates with a target protein by forming a sequence-directed covalent bond with a HUH like tag that is fused to the protein of interest. Examples of useful applications for such an approach is the generation of DNA-barcoded antibodies which can be used for analyte detection. Other applications for such DNA barcoded proteins are in the detection and characterization of patient specific antibodies against both foreign proteins and self. Additional usage for DNA-barcoded proteins are in the performance mapping of protein-protein interactions, or as tags to track the localization of the barcoded protein when exposed to a complex cellular environment (e.g. injected within an animal).

In other embodiments, the approach can be used to generate ssDNA within cells that can be used as a method of cellular barcoding that can be exploited to label a large population of single cells each with a unique identifier. These identifiers can then be used to uniquely track individual proteins and can be read through conventional approaches such as single cell sequencing based methodologies which are well known to the field.

The generated ssDNA can also be used as a reporter of transcriptional activity by placing the RNA that is reverse transcribed under the control of a promoter of interest. When the RNA is expressed, this will then lead to it being converted into ssDNA in the cell which can be read out using DNA sequencing based approaches, skipping the need to perform an in vitro reverse transcription step.

In conclusion, the present disclosure can be used to increase the efficiency and scale at which one can produce mutant cell lines. Since the methods and systems of the disclosure are effective in mammalian cells, they could be used for the modeling of human disease in cell culture and in vivo in mouse models. The methods and systems of the disclosure decrease the cost of producing these models, as well as the time required to produce these models, compared to current methodologies. In addition, the methods and systems of the disclosure also decrease the effort needed to test for potential therapeutic benefit of many antisense oligonucleotides or aptamers in parallel in a particular disease model. Furthermore, the methods and systems of the disclosure can be used in a therapeutic context for ex vivo genome editing of human cells, or gene correction in vivo in patients.

We claim:

1. A method of producing a single stranded DNA oligonucleotide in vivo, comprising
introducing a DNA construct encoding a sequence of interest into a cell, wherein the sequence of interest is operably linked to a promoter sequence recognizable by the cell's endogenous RNA polymerase machinery;
introducing a reverse transcriptase fused to a nuclear localization signal and a RNA binding protein into the cell;
wherein the sequence of interest is fused to a motif that is recognized by the RNA binding protein after being transcribed;
thereby producing the single stranded DNA oligonucleotide.

2. The method of claim 1, further comprising introducing a DNA cleaving enzyme into the cell to cleave a target locus, wherein the single stranded DNA oligonucleotide repairs the DNA by homology-directed repair (HDR).

3. The method of claim 2, wherein the DNA cleaving enzyme is Cas9, a Zinc Finger enzyme, an Activator-Like Effector Nuclease (TALEN) enzyme, or any one of the CRISPR-associated DNA cleaving enzymes.

4. The method of claim 1, wherein the reverse transcriptase is a human immunodeficiency virus reverse transcriptase (HIV RT).

5. The method of claim 1, wherein the RNA binding protein is a MS2 coat protein (MCP).

6. The method of claim 1, wherein the RNA binding protein is a PP7 coat protein (PCP).

7. The method of claim 1, wherein the RNA binding protein is fused to the N-terminus, the C-terminus, or both termini of the reverse transcriptase.

8. The method of claim 1, wherein the DNA construct further comprises an HIV terminator-binding site (HTBS).

9. The method of any of claim 1, wherein the motif is a MS2 RNA motif.

10. The method of claim 1, wherein the motif is a PP7 RNA motif.

11. The method of claim 1, wherein the cell is a mammalian cell.

12. The method of claim 1, further comprising DNA barcoding biomolecules within cells using the single stranded DNA oligonucleotide.

13. The method of claim 12, wherein the biomolecule is a protein.

14. The method of claim 1, wherein a plurality of different DNA constructs are introduced into a population of cells, such that each cell receives a single DNA construct, to thereby produce a plurality of ssDNA oligonucleotides.

15. The method of claim 14, further comprising screening the plurality of ssDNA oligonucleotides to identify ssDNA oligonucleotides having one or more desired property.

16. The method of claim 14, further comprising screening the plurality of ssDNA oligonucleotides to identify one or more ssDNA oligonucleotide that functions as an antisense oligonucleotide to knockdown a target gene of interest.

17. The method of claim 14, further comprising screening the plurality of ssNDA oligonucleotides to identify one or more ssDNA oligonucleotide that functions as an aptamer to bind to a target protein of interest.
